(19) United States
(12) Patent Application Publication
(10) Pub. No.: US 2022/0074873 A1
(43) Pub. Date: Mar. 10, 2022

(54) RADIOLOGICAL IMAGE PROCESSING DEVICE, RADIOLOGICAL IMAGE PROCESSING METHOD, AND RADIOLOGICAL IMAGE PROCESSING PROGRAM

(71) Applicant: SHIMADZU CORPORATION, Kyoto-shi (JP)

(72) Inventors: Takahiro MIYAJIMA, Kyoto-shi (JP); Junya YAMAMOTO, Kyoto-shi (JP); Kazuyoshi NISHINO, Kyoto-shi (JP)

(21) Appl. No.: 17/416,501

(22) PCT Filed: Oct. 17, 2019
(86) PCT No.: PCT/JP2019/041000

§ 371 Date: Jun. 20, 2021

(30) Foreign Application Priority Data
Dec. 21, 2018 (JP) 2018-239838

(51) Int. Cl. G01N 23/046; G06T 7/00; G06T 7/70

(52) U.S. Cl. CPC G01N 23/046; G06T 7/0012; G06T 2207/30204; G06T 2207/10116; G06T 7/70

(57) ABSTRACT

[Problem] To provide a radiographic image processing technique capable of detecting a metal marker from a radiographic image at high speed and with a high degree of accuracy.

[Solution] The above-described problem is solved by a radiographic image processing apparatus including: an acquisition unit configured to acquire a radiographic image reflecting a plurality of marker; a generation unit configured to generate a low-resolution image in which the resolution of the radiographic image has been reduced; a position identification unit configured to identify respective positions of a plurality of markers in the low-resolution image based on a characteristic of the plurality of markers; and a position estimation unit configured to estimate positions of the plurality of markers in the radiographic image by searching for positions on the radiographic image corresponding to the respective positions of the plurality of markers in the low-resolution image.

### TECHNICAL FIELD

The present invention relates to radiographic image processing apparatus, a radiographic image processing method, and a radiographic image processing program.

### BACKGROUND OF THE INVENTION

As a radiographic image processing technique, for example, the following technique is known. In this technique, X-rays are emitted from an X-ray tube to a subject, and X-rays transmitted through the subject are detected by a flat-panel X-ray detector (hereinafter referred to as “FPD”), thereby acquiring a projected image. At this time, the first, second, and third cameras capture an optical image of a marker disposed on a monitoring plate to obtain the image. Then, a three-dimensional position calculation unit calculates the three-dimensional position of the X-ray tube and the FPD, based on the respective acquired images. A reconstruction calculation unit generates a tomographic image or the like based on the group of projected images and the measured three-dimensional positions (see, e.g., Patent Document 1).

There also is the following technique. In this technique, a series of radiographic images are captured in a state in which a marker is reflected together with a subject in the imaging field of view. Based on the marker images reflected in the respective radiographic images, it is possible to recognize how much the imaging system deviates from the ideal position. Based on this recognition, the image correction is performed (see, e.g., Patent Document 2).

### PRIOR ART DOCUMENT

### Patent Document

Patent Document 1: Japanese Unexamined Patent Application Publication No. 2006-181252
Patent Document 2: Japanese Unexamined Patent Application Publication No. 2013-17675

### SUMMARY OF THE INVENTION

### Problems to be Solved by the Invention

For example, an X-ray tomographic plane examination apparatus using an X-ray Tomosynthesis detects an image of the tomographic plane by synthesizing a plurality of image data acquired by one imaging. At this time, when synthesizing a plurality of image data, the position of the X-ray tube emitting the X-rays needs to be calculated. As a premise, it is required to detect a metal marker embedded in a phantom to be reflected together with a subject.

However, in order to detect the metal marker from the captured radiographic image, it is required to scan the region of interest in the captured image to repeat the binarization, so that an enormous amount of processing and time are required. Further, in a case where the position of the metal marker is detected in the image, the X-ray tube position estimation result greatly changes with the accuracy of less than one pixel, so that the accuracy of detecting the marker position is also required.

In one aspect, the present invention provides a radiographic image processing technique capable of detecting a metal marker from a radiographic image at high speed and with a high degree of accuracy.

### Means for Solving the Problem

A radiographic image processing apparatus according to one aspect of the present invention, includes:

an acquisition unit configured to acquire a radiographic image reflecting a plurality of markers:

a generation unit configured to generate a low-resolution image in which a resolution of the radiographic image has been reduced;

a position identification unit configured to identify respective positions of the plurality of markers in the low-resolution image, based on a characteristic of the plurality of markers; and

a position estimation unit configured to estimate positions of the plurality of markers in the radiographic image, by searching for positions on the radiographic image corresponding to the respective positions of the plurality of markers in the low-resolution image.

The above-described radiographic image processing apparatus may further include:

a search unit configured to search for a region of interest reflecting the plurality of markers in the low-resolution image, based on the characteristic of the plurality of markers.

The above-described search unit may narrow down a scan region with respect to the low-resolution image in a stepwise manner, based on the characteristic of the plurality of markers.

The above-described search unit may identify a temporary region of interest including a region reflecting the plurality of markers in the low-resolution image and identifies the region of interest reflecting the plurality of markers from the temporary region of interest based on the characteristic of the plurality of markers.

The above-described position identification unit may identify respective barycentric coordinates of the plurality of markers included in the region of interest as the respective positions of the plurality of markers in the low-resolution image, based on the characteristic of the plurality of markers.

A radiographic image processing method to be performed by a radiographic image processing apparatus according to one aspect of the present invention, includes:

acquiring a radiographic image reflecting a plurality of markers;

generating a low-resolution image in which a resolution of the radiographic image has been reduced;

identifying respective positions of the plurality of markers in the low-resolution image, based on the characteristic of the plurality of markers; and

estimating positions of the plurality of markers in the radiographic image by searching for positions on the radiographic image corresponding to the respective positions of the plurality of markers in the low-resolution image.

A radiographic image processing program according to one aspect of the present invention is configured to making a computer execute processing, the processing including:

acquiring a radiographic image reflecting a plurality of markers;

generating a low-resolution image in which a resolution of the radiographic image has been reduced;

identifying respective positions of the plurality of markers in the low-resolution image, based on the characteristic of the plurality of markers; and

estimating positions of the plurality of markers in the radiographic image by searching for positions on the radiographic image corresponding to the respective positions of the plurality of markers in the low-resolution image.

### Effects of the Invention

According to one aspect of the present invention, a metal marker can be detected from a radiographic image at high speed and with a high degree of accuracy.

### BRIEF DESCRIPTION OF THE DRAWINGS

FIG. 1 is a schematic diagram showing an entire configuration of a radiographic image capturing apparatus according to an embodiment.

FIG. 2 is a schematic diagram showing one example of a phantom used in this embodiment.

FIG. 3 is a block diagram showing a configuration example of the radiographic image processing apparatus of this embodiment.

FIG. 4 is a flowchart showing the entire processing of a control unit of the radiographic image capturing apparatus of this embodiment.

FIG. 5 is a flowchart showing the detail of the marker position estimation processing (S1) of this embodiment.

FIG. 6 is a diagram for explaining the processing of S12 in FIG. 5.

FIG. 7 is a diagram for explaining the processing of S13 in FIG. 5.

FIG. 8 is a diagram for explaining the processing of S14 in FIG. 5.

FIG. 9 is a diagram for explaining the processing of S15 in FIG. 5.

FIG. 10 is a diagram for explaining the processing of S16 in FIG. 5.

### EMBODIMENTS FOR CARRYING OUT THE INVENTION

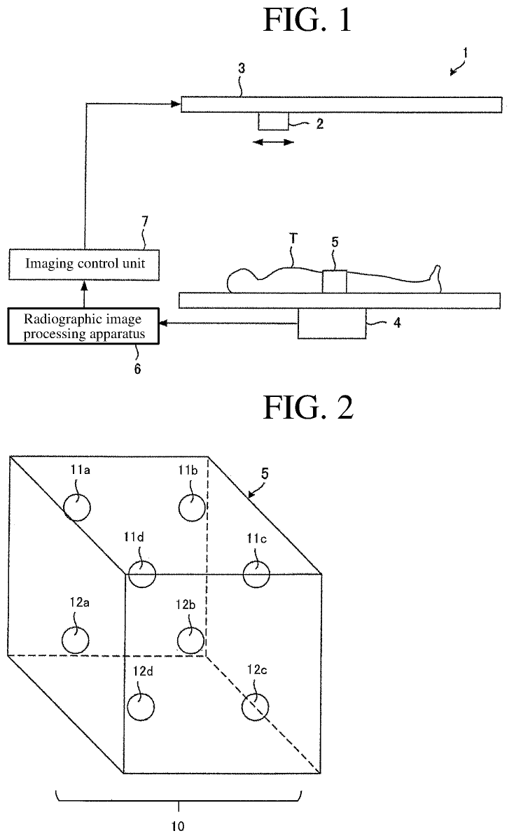

FIG. 1 is a schematic diagram illustrating the entire configuration of a radiographic image capturing apparatus of this embodiment. The radiographic image capturing apparatus 1 is an apparatus for performing radiographic imaging, such as, e.g., tomosynthesis imaging, for medical use. This apparatus 1 acquires a plurality of image data by imaging a subject T while changing the position of an X-ray tube 2, which is a radiation source. Specifically, the radiographic image capturing apparatus 1 is provided with an X-ray tube 2, a position change mechanism 3, a detector 4, a phantom 5, a radiographic image processing apparatus 6, an imaging control unit 7, and the like.

When a high voltage is applied based on the signal from the imaging control unit 7, the X-ray tube 2 generates radiation (X-rays) and emits the radiation toward the detector 4. The X-ray tube 2 is movably held by the position change mechanism 3. The position change mechanism 3 changes the position of the X-ray tube 2 based on the signal from the imaging control unit 7.

The detector 4 is a flat panel X-ray detector (Flat Panel Detector: FPD). This detector 4 is arranged to face the X-ray tube 2, and converts the captured image by the radiation emitted from the X-ray tube 2 into image data. That is, the detector 4 converts the radiation to an electric signal, reads the converted electric signal as a signal of the image, and outputs the signal of the image to the radiographic image processing apparatus 6. Note that the detector 4 is provided with a plurality of conversion elements (not shown) and pixel electrodes arranged on the plurality of conversion elements (not shown). Further, the plurality of conversion elements and pixel electrodes are arranged at a predetermined period (pixel pitch).

The phantom 5 is also referred to as a calibration phantom, and has a configuration in which metallic spheres are arranged at the center of a rectangular parallelepiped made of, for example, acrylic resin or the like. The phantom 5 is arranged between the X-ray tube 2 and the detector 4, and is imaged together with the subject T to estimate the position of the X-ray tube 2.

The radiographic image processing apparatus 6 is an apparatus for processing the signal of the image acquired by the detector 4. The configuration of the radiographic image processing apparatus 6 will be described later.

FIG. 2 is a schematic diagram showing an example of a phantom used in this embodiment. The phantom 5 is made of resin or the like, and has a plurality of metal markers 11a, 11b, 11c, 11d, 12a, 12b, 12c, 12d therein. The metal marker is made of metal, such as, e.g., aluminum, gold, lead, and tungsten. The metal marker 11a and the metal marker 12a are arranged and paired in a distance in the near and far direction with respect to the detector 4. The metal marker 11b and the metal marker 12b are arranged and paired in a distance in the near and far direction with respect to the detector 4. The metal marker 11c and the metal marker 12c are paired and arranged in a distance in the near and far direction with respect to the detector 4. The metal marker 11d and the metal marker 12d are paired and arranged in a distance in the near and far direction with respect to the detector 4.

Hereinafter, the metal marker may be referred to as a “marker”. The metal markers (or markers) 11a, 11b, 11c, 11d, 12a, 12b, 12c, 12d are collectively referred to as metal markers (or markers) 10.

Here, the paired metal markers are arranged at least 70 mm apart from each other in the near and far direction. Further, the metal markers constituting the pair are arranged at positions that do not overlap when viewed in the near and far direction (when the phantom 5 is viewed in a plan).

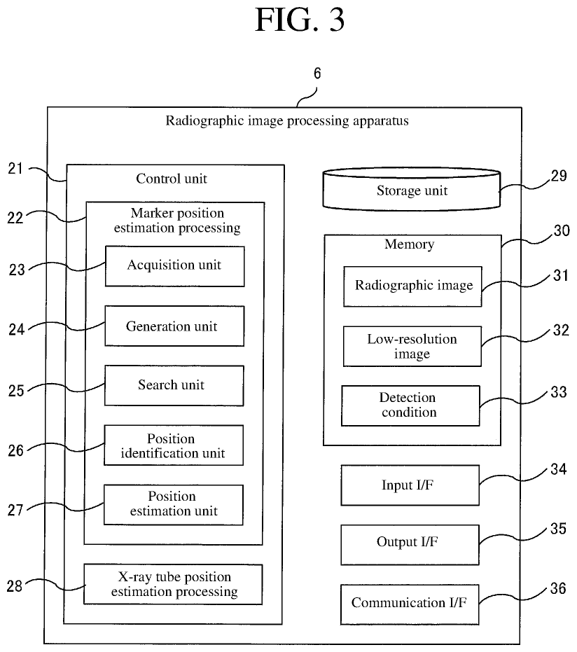

FIG. 3 is a block diagram showing a configuration example of the radiographic image processing apparatus of this embodiment. The radiographic image processing apparatus 6 includes a control unit 21, a storage unit 29, a memory 30, an input interface 34, an output interface 35, and a communication interface 36. Hereinafter, the interface is referred to as “I/F”. The control unit 21, the storage unit 29, the memory 30, the input l/F 34, the output l/F 35, and the communication I/F 36 are connected to each other by a bus (not shown) that transfers command signals or data signals.

The control unit 21 is, for example, a processor (not shown), such as, e.g., a CPU (Central Processing Unit), a GPU (Graphics Processing Unit), or an FPGA (Field-Programmable Gate Array) configured for image processing. The control unit 21 controls the entire operation of the radiographic image processing apparatus 6 and performs the image processing.

The storage unit 29 is a large-capacity storage device, such as, e.g., a hard disk drive and an SSD (Solid State Drive), and stores a radiographic image 30 acquired by the detector 4. The storage unit 29 stores the information on the detection condition 33 of the marker used in this embodiment. In the storage unit 29, an operating system (OS) and a program related to radiographic image processing (including a program associated with this embodiment) are installed.

The memory 30 is a working storage region used by the control unit 21 to perform predetermined processing or to display data on a screen. The memory 30 is a volatile storage device, such as, e.g., a RAM (Random Access Memory), but may be a non-volatile flash memory depending on the specification.

The input I/F 34 is, for example, an interface to which an input device (not shown), such as, e.g., a keyboard and a control panel, is connected. The detection condition 33 of the marker 10 can be set via the input device. The output I/F 35 is an interface to which, for example, a display device, such as, e.g., a touch panel, and/or an output device (not shown), such as, e.g., a printer, is connected. The communication I/F 36 is an interface for communicating with other devices, such as, e.g., the detector 4 and the imaging control unit 7.

Next, the processing performed by the control unit 21 will be described. The control unit 21 generally performs marker position estimation processing 22 and X-ray tube position estimation processing 28 in this embodiment. The marker position estimation processing 22 is processing for estimating the position of the reflected marker 10 from the captured radiographic image. When performing the marker position estimation processing 22, the control unit 21 reads out and executes the program of this embodiment stored in the storage unit 29. With this, the control unit 21 functions as an acquisition unit 23, a generation unit 24, a search unit 25, a position identification unit 26, and a position estimation unit 27. At this time, the control unit 21 reads out the detection condition 33 stored in the storage unit 29 and places it in the memory 30.

The acquisition unit 23 acquires a radiographic image 31 reflecting a plurality of markers 10 via the communication I/F 35 or stored in the storage unit 29, and places it in the memory 31.

The generation unit 24 generates a low-resolution image 32 in which the resolution of the radiographic image 31 has been reduced and arranges it in the memory 30.

The search unit 25 searches for a region of interest reflecting the plurality of markers 10 in the low-resolution image 32 based on the characteristic of the plurality of markers set in the detection condition 33. Here, the region of interest represents a predetermined region selected for the image analysis from the low-resolution image 32. The search unit 25 can narrow down the scan region with respect to the low-resolution image 32 in a stepwise manner based on the characteristic of the plurality of markers 10. Based on the characteristic of the plurality of markers, the search unit 25 may identify a temporary region of interest including a region reflecting the plurality of markers 10 in the low-resolution image 32, and may identify the region of the interest reflecting one or a plurality of markers from the temporary region of interest.

The position identification unit 26 identifies the respective positions of the plurality of markers 10 in the low-resolution image 32 based on the characteristic of the plurality of markers set in the detection condition 33. More specifically, the position identification unit 26 identifies the respective positions of the plurality of markers 10 included in the region of interest in the low-resolution image 32, based on the characteristic of the plurality of markers 10 set in the detection condition 33. Based on the characteristic of the plurality of markers 10, the position identification unit 26 identifies the respective barycentric coordinates of the plurality of markers 10 included in the region of interest as the respective positions of the plurality of markers 10 in the low-resolution image 32.

The position estimation unit 27 estimates the positions of the plurality of markers 10 in the radiographic image 31 by searching for positions on the radiographic image 31 corresponding to the respective positions of the plurality of markers 10 in the low-resolution image 32.

The X-ray tube position estimation processing 28 identifies the above-described pairs in the vertical direction in the phantom 5, based on the position and the area of the marker reflected in the radiographic image 31 estimated by the marker position estimation processing 22. The X-ray tube position estimation processing 28 estimates the position of the X-ray tube based on the position coordinate of the marker identified as a pair.

The program according to this embodiment may be executed not only by the radiographic image processing apparatus 6 but also by an information processing device, such as, e.g., a computer. The program in this embodiment may be installed on the computer from a telecommunication network or a recording medium.

A recording medium including such a program is configured by a removable media that is distributed separately from the device body to the user to provide the program to each user. The recording medium may also be configured by a recording medium or the like provided to each user in a condition in which it is incorporated in the device main body in advance.

In this specification, the step describing a program recorded in the recording medium includes processing performed in time series in the order. Further, this step includes the processing that is executed in parallel or individually, although not necessarily executed in chronological order.

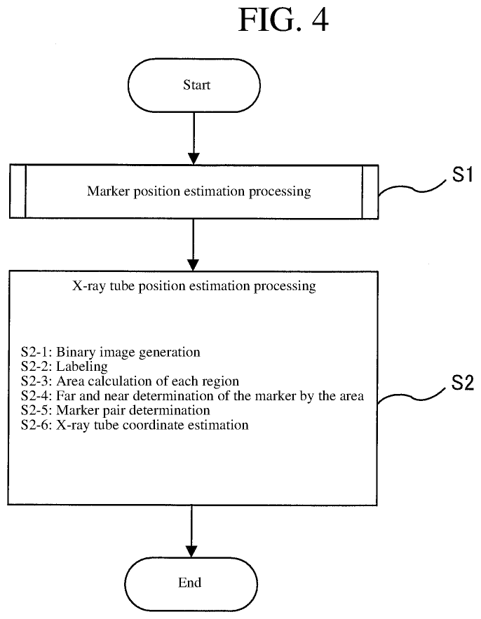

FIG. 4 is a flowchart showing the entire processing of the control unit of the radiographic image capturing apparatus in this embodiment. The control unit 21 performs the marker position estimation processing (S1). The marker position estimation processing (S1) is processing for estimating the position of the marker 10 reflected in the captured radiographic image 31 from the captured radiographic image 31. The detailed processing of S1 will be described later.

Next, the control unit 21 performs X-ray tube position estimation processing (S2). In the X-ray tube position estimation processing (S2), the following processing is executed in order. The processing includes: binary image generation processing (S2-1); labeling processing (S2-2); area calculation processing (S2-3) of each region; far and near determination processing (S2-4) of a marker by an area; marker pair determination processing (S2-5); and X-ray tube coordinate estimation processing (S2-6).

In the binary image generation processing (S2-1), the control unit 21 generates a binarized radiographic image, based on the signal of the image detected by the detector 4.

In the labeling processing (S2-2), the control unit 21 labels each of the metal markers 11a-11d, 12a-12d for which the positions were estimated by the marker position estimation processing (S1) in the radiographic image to distinguish them from each other.

The area calculation processing (S2-3) of each region is processing in which the control unit 21 calculates the area of each of the plurality of metal markers 11a to 11d and 12a to 12d in the labeled radiographic image. Here, the control unit 21 also calculates the average value of the maximum value and the minimum value of the calculated areas.

In the far and near determination processing (S2-4) of the marker by an area, the control unit 21 determines that the metal markers 11a to 11d in the radiographic image having an area larger than the calculated average value as a threshold is relatively far from the detector 4 (positioned at the upper portion within the phantom 5 in FIG. 2) and classifies them as a first group. The control unit 21 determines that the metal markers 12a to 12d in the radiographic image having an area smaller than the average value are relatively close to the detector 4 (positioned at the lower portion within the phantom 5 in FIG. 2) and classifies them as a second group.

In the marker pair determination processing (S2-5), the control unit 21 classifies the plurality of metal markers 11a-11d, 12a-12d based on the relative position on the x-y coordinate plane of the plurality of metal markers 11a-11d, 12a-12d for each classified group. Then, the control unit 21 selects the metal markers 11a-11d of the first group and the metal markers 12a-12d of the second group, which match the relative position, as pairs.

Specifically, the control unit 21 selects, for example, one of the following pairs as the metal markers in which the relative position matches. That is, the control unit 21 selects one of the pair of the metal markers 11a and the metal marker 12a, the pair of the metal markers 11b and the metal marker 12b, the pair of the metal marker 11c and the metal marker 12c, and the pair of the metal marker 11d and the metal marker 12d.

Note that in the phantom 5, as a plurality of pairs of metal markers arranged in a distance in the near and far direction with respect to the detector 4, here, the four pairs are exemplified as the configurable number, but the present invention is not limited thereto. Even considering that some metal markers are not reflected in the captured image due to, for example, tilting of the phantom 5, in order to estimate the X-ray tube position, it is sufficient that at least two pairs of metal markers are provided. Further note that the marker is not limited to a metal one, and any material may be used as long as the absorption amount of X-rays is large.

In the X-ray tube coordinate estimation processing (S2-6), the control unit 21 estimates the position of the X-ray tube 2, based on the position coordinate of the paired and selected metal markers 11a-11d, 12a-12d. Now a three-dimensional space including the X-ray tube 2, the metal markers 11a, 12a, and the metal markers 11a, 12a in the radiographic image is assumed. At this time, the position coordinate of the position S of the X-ray tube 2 is defined as (x, y, Sd). Further, the position coordinate of the position of the metal marker 11a is defined as (Pa, Pb, Pd+Ps). The position coordinate of the position of the metal marker 12a is defined as (Pa, Pb, Pd). The position coordinate of the position of the metal marker 11a in the radiographic image is defined as (a1, b1, 0). The position coordinate of the position of the metal marker 12a in the radiographic image is defined as (a2, b2, 0).

Note that x is a coordinate of the X-ray tube 2 in the X-direction. Also, y is a coordinate of the X-ray tube 2 in the Y-direction. In addition, Pa is a coordinate of the metal marker 11a, 12a in the X-direction. Pb is a coordinate of the metal markers 51a and 52a in the Y-direction. Sd is a distance (SID: Source Image receptor Distance) in the Z-direction from the detector 4 to the X-ray tube 2. Further, Pd is a distance in the Z-direction from the detector 4 to the metal marker 12a. Further, Ps is a distance in the Z-direction between the metal markers 11a and 12a to each other.

The X-ray tube 2, the metal markers 11a and 12a, and the metal markers 11a and 12a in the radiographic image are in the relation of externally dividing points. Therefore, from this relation, the position coordinate of the position S of the X-ray tube 2 is derived from the following Expressions (1) and (2).

x={a1*(1−β)−a2*(1−α)}/(β−α) (1)

y={b1*(1−β)−b2*(1−α)}/(β−α) (2)

where,

α=(Pd+Ps)/(Pd+Ps−Sd)

β=Pd/(Pd−Sd)

With this, even if the radiographic image capturing apparatus 1 does not have a mechanism to measure the absolute position, it is possible to estimate the position of the X-ray tube by the positional relation of the plurality of markers in the radiographic image.

Next, the marker position estimation processing (S1) will be described in detail.

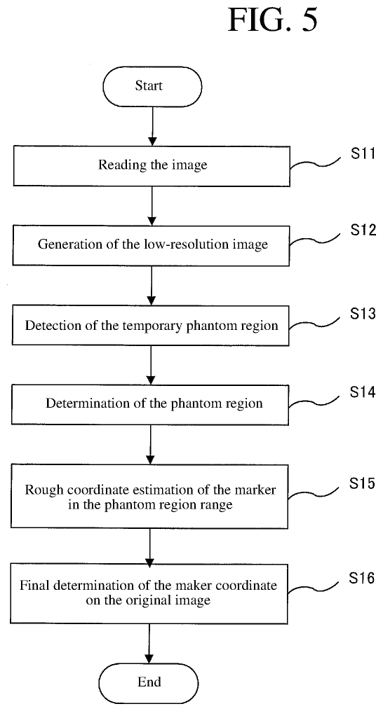

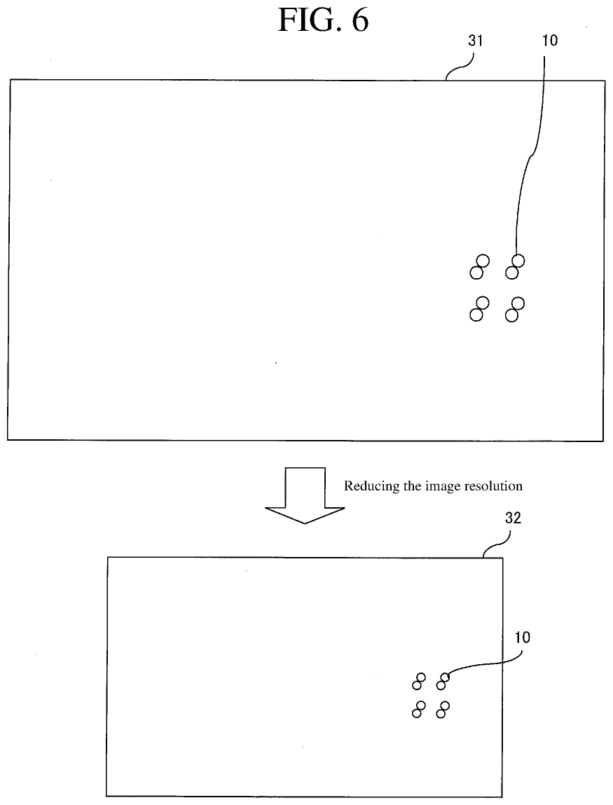

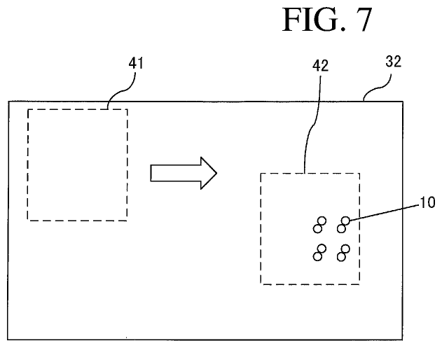

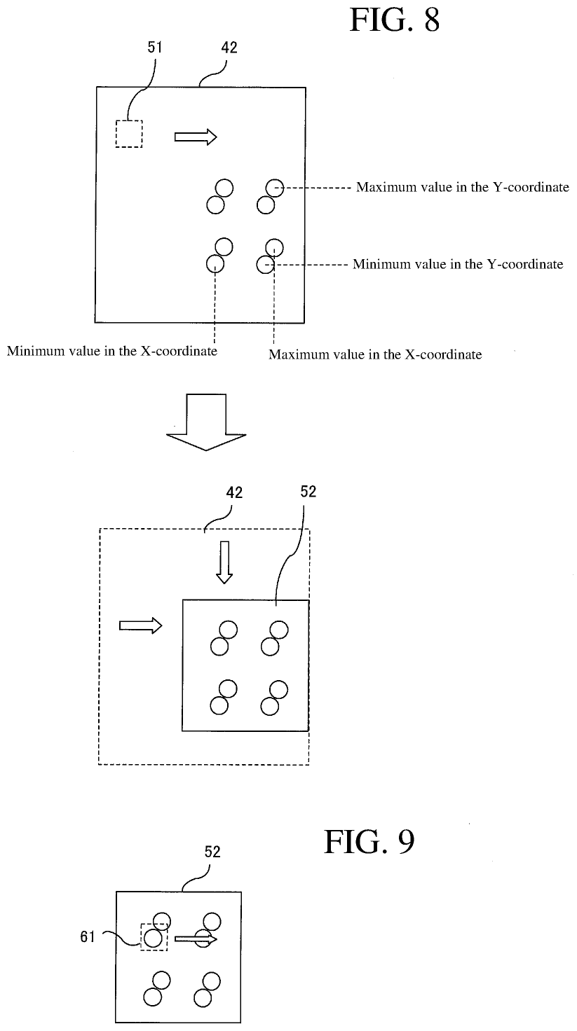

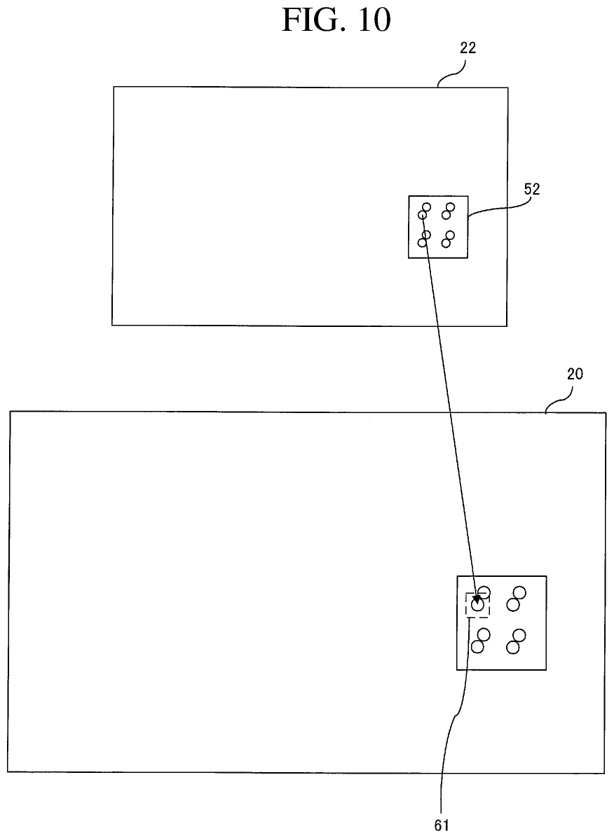

FIG. 5 is a flowchart showing the detail of the marker position estimation processing (S1) in this embodiment. FIG. 6 is a diagram for explaining the processing of S12 in FIG. 5. FIG. 7 is a diagram for explaining processing of S13 in FIG. 5. FIG. 8 is a diagram for explaining processing of S14 in FIG. 5. FIG. 9 is a diagram for explaining processing of S15 in FIG. 5. FIG. 10 is a diagram for explaining processing of S16 in FIG. 5.

In S1, the control unit 21 reduces the processing time required to estimate the position of the marker by narrowing down the scan range of the radiographic image with the reduced resolution in a stepwise manner. Also, since the X-ray tube coordinate changes with the accuracy of less than one pixel, the coordinate of the final marker is estimated using the radiographic image of the original resolution. Note that it is assumed that the data of the captured image (radiographic image) reflecting the subject T and the phantom 5 acquired by the detector 4 has been stored in advance in the storage unit 29.

First, as the acquisition unit 23, the control unit 21 reads out the radiographic image 31 stored in the storage unit 29 and arranges it in the memory 30 (S11).

Next, as shown in FIG. 6, as the generation unit 24, the control unit 21 reduces the resolution of the read radiographic image 31 to generate a low-resolution image 32 having a reduced amount of information (S12). As a method to reduce the resolution of the radiographic image 31, for example, the resolution of the image may be reduced by integrating the pixels by applying the average value filter to the pixel block. Alternatively, for example, the resolution may be reduced by extracting one pixel of the characteristic point from the pixel block or may simply subtract pixels. Alternatively, the filter is not necessarily a mean-valued filter and may be a filter capable of smoothing the pixels.

The degree of reduction in the resolution of the radiographic image 31 may be arbitrarily set by the operator by, for example, a control panel or the like, or may be set to a predetermined value in advance.

Next, as the search unit 25, the control unit 21 detects the rough position of the phantom (the region where a maker may be present, the region being referred to as a temporary phantom region) from the low-resolution image 32. Here, as shown in FIG. 7, the control unit 21 scans the region of interest 41 within the low-resolution image 32 for binarizing and labeling. The binarizing denotes the processing for binarizing each pixel within the image region in the scan range, based on a preset threshold of the pixel value. The labeling denotes the processing in which, when the binarized pixel and the neighboring binarized pixel are equal in value, grouping is performed, and the closed region is determined to be the same object by repeating the grouping, and the closed region is distinguished for each object. In this instance, in particular, the control unit 21 performs labeling processing on each of the plurality of metal markers 11a-11d, 12a-12d in the low-resolution image 32 to distinguish the image of the individual metal marker from the others, and labels them.

When performing the labeling, the control unit 21 detects and labels the metal marker from the low-resolution image 32 based on the detection condition 33. The detection condition 33 defines the characteristic of the metal marker reflected in the low-resolution image 32, and is, for example, the circularity and/or the area of the marker in the low-resolution image 32. For example, when there exit the largest number of labeled objects whose circularity and/or area satisfy a predetermined condition (threshold value), the control unit 21 sets the region specified by the position 42 of the region of interest as a phantom region.

Then, as the search unit 25, the control unit 21 determines the phantom position to be estimated based on the temporary phantom region 42 (S14). That is, as shown in FIG. 8, in the temporary phantom region 42 in the low-resolution image 32, for example, the control unit 21 scans the region of interest 51 having a size larger than the marker (e.g., 1.5 times the size of the marker) for binarizing and labeling. The control unit 21 then detects the labeled object satisfying the detection condition 33 from the labeled object. The control unit 21 acquires objects having the maximum value and the minimum value in the X-coordinate and Y-coordinate among the labeled objects satisfying the detection condition 33. The control unit 21 determines the region of a predetermined range centered on the average value of the maximum value and the minimum value in the X-coordinate and Y-coordinate and minimum value as a phantom region 52.

Then, as the position identification unit 26, the control unit 21 estimates the rough coordinate of the markers in the range of the phantom region 52 (S15). Here, as shown in FIG. 9, the control unit 21 scans the region of interest 51 having a size larger than, for example, the marker (for example, 1.5 times the size of the marker) in the determined phantom region 52, and performs binarizing and labeling. The control unit 21 then detects the labeled object satisfying the detection condition 33 from the labeled objects. The control unit 21 records the respective barycentric coordinates of the labeled objects satisfying the detection condition 33.

As the position estimation unit 27, the control unit 21 makes a final determination of the coordinate of each marker on the original radiographic image 31 prior to the resolution reduction (S16). Here, as shown in FIG. 10, the control unit 21 performs the following processing in the original radiographic image 31 prior to the resolution reduction. That is, the control unit 21 sets a region of interest 61 having a size larger than the marker (e.g., 1.5 times the size of the marker) centered on the coordinate corresponding to the barycentric coordinate of each object labeled in the low-resolution image 32. Then, the control unit 21 performs binarizing within the region of interest 61 and calculates the coordinate of the final marker.

According to this embodiment, in the radiographic image reflecting the marker, the position of the phantom in which makers are embedded is temporarily identified from the radiographic image reduced in resolution. Then, the search range is narrowed down by using the region specified as the position of the temporary phantom as the region of interest, and the rough position of each marker is specified. This allows the estimation of the coordinate of the final marker in the original radiographic image prior to the resolution reduction. As a result, the processing time required for estimating the position of markers can be shortened by narrowing down the scan range of the radiographic image in which the resolution has been reduced in a stepwise manner. Also, since the estimation of the coordinate of the final marker is performed using the radiographic image of the original resolution, it is possible to cope with the change in the X-ray tube coordinate with the accuracy of less than one pixel.

Note that in the above, labeling is performed to detect labeled objects satisfying the detection condition 33 from labeled objects. However, the detection condition 33 may be set for each labeling, or may be the same extraction condition.

Note that, in the above-described embodiment, an image acquired by tomosynthesis has been described as an example as the radiographic image, but the present invention is not limited thereto, and an image acquired by tomography photographing, such as, e.g., CT (Computed Tomography), may be used. Alternatively, the image applied to this embodiment may be, for example, an MRI (magnetic resonance imaging) image or another medical image.

As described above, a radiographic image processing apparatus (for example, a radiographic image processing apparatus 6) includes:

an acquisition unit (e.g., the acquisition unit 23) configured to acquire a radiographic image (e.g., the radiographic image 31) reflecting a plurality of markers:

a generation unit (e.g., the generation unit 24) configured to generate a low-resolution image (e.g., the low-resolution image 32) in which a resolution of the radiographic image has been reduced;

a position identification unit (e.g., the position identification unit 26) configured to identify respective positions of the plurality of markers in the low-resolution image, based on a characteristic (e.g., the detection condition 33) of the plurality of markers; and

a position estimation unit (e.g., the position estimation unit 27) configured to estimate positions of the plurality of markers in the radiographic image, by searching for positions on the radiographic image corresponding to the respective positions of the plurality of markers in the low-resolution image.

With this configuration, it is possible to detect the metal markers from the radiographic image at high speed and with a high degree of accuracy. In other words, since the scan range of the radiographic image in which the resolution has been reduced can be narrowed down in a stepwise manner, the processing time required for estimating the positions of the markers can be shortened. Further, the X-ray tube coordinate changes with the accuracy of less than one pixel, but the final estimation of the coordinates of the markers is performed using the radiographic image of the original resolution. Therefore, it is possible to estimate the coordinates of the markers with a high degree of accuracy, and as a result, it is possible to estimate the X-ray tube coordinate with a high degree of accuracy.

The radiographic image processing apparatus (e.g., the radiographic image processing apparatus 6) is further provided with:

a search unit (e.g., the search unit 25) configured to search for a region of interest reflecting the plurality of markers in the low-resolution image, based on the characteristic of the plurality of markers.

With this configuration, a plurality of marker regions of interest in the low-resolution image can be searched.

The search unit (e.g., the search unit 25) narrows down a scan region with respect to the low-resolution image in a stepwise manner, based on the characteristic of the plurality of markers.

With this configuration, it is possible to narrow down the region in which the markers exist.

The search unit (e.g., the search unit 25) identifies a temporary region of interest (e.g., the temporary phantom region 42) including a region reflecting the plurality of markers in the low-resolution image and identifies the region of interest (e.g., the phantom region 52) reflecting the plurality of markers from the temporary region of interest based on the characteristic of the plurality of markers.

With this configuration, the position in which the phantom exists can be estimated from the rough phantom region.

The position identification unit (e.g., the position identification unit 26) identifies respective barycentric coordinates of the plurality of markers included in the region of interest as the respective positions of the plurality of markers in the low-resolution image, based on the characteristic of the plurality of markers.

With this configuration, although the X-ray tube coordinate changes with the accuracy of less than one pixel, the estimation of the coordinate of the final marker is performed using the radiographic image of the original resolution. Therefore, it is possible to estimate the coordinate of the marker with a high degree of accuracy, and as a result, it is possible to estimate the X-ray tube coordinate with a high degree of accuracy.

Further, a radiographic image processing method to be performed by a radiographic image processing apparatus according to this embodiment, includes:

acquiring a radiographic image (e.g., the radiographic image 31) reflecting a plurality of markers (e.g., S11 in FIG. 5);

generating a low-resolution image (e.g., the low-resolution image 32) in which a resolution of the radiographic image has been reduced (e.g., S12 in FIG. 5);

identifying respective positions of the plurality of markers in the low-resolution image, based on the characteristic (e.g., the detection condition 33) of the plurality of markers (e.g., S15 in FIG. 5); and

estimating positions of the plurality of markers in the radiographic image by searching for positions on the radiographic image corresponding to the respective positions of the plurality of markers in the low-resolution image (e.g., S16 in FIG. 5).

With this configuration, the metal markers can be detected from the radiographic image at high speed and with a high degree of accuracy. In other words, since the scan range of the radiographic image in which the resolution has been reduced can be narrowed down in a stepwise manner, the processing time required for estimating the position of the markers can be shortened. In addition, although the X-ray tube coordinate changes with the accuracy of less than one pixel, the estimation of the coordinate of the final marker is performed using the radiographic image of the original resolution, so that the coordinate of the marker with a high degree of accuracy can be estimated, resulting in the estimation of the high-precision X-ray tube coordinate.

Further, the radiographic image processing program according to this embodiment makes a computer execute the processing comprising:

acquiring a radiographic image (e.g., the radiographic image 31) reflecting a plurality of markers (e.g., S11 in FIG. 5);

generating a low-resolution image (e.g., the low-resolution image 32) in which a resolution of the radiographic image has been reduced (e.g., S12 in FIG. 5);

identifying respective positions of the plurality of markers in the low-resolution image, based on the characteristic (e.g., the detection condition 33) of the plurality of markers (e.g., S15 in FIG. 5); and

estimating the positions of the plurality of markers in the radiographic image by searching for positions on the radiographic image corresponding to the respective positions of the plurality of markers in the low-resolution image (e.g., S16 in FIG. 5).

With this configuration, the metal markers can be detected from the radiographic image at high speed and with a high degree of accuracy. In other words, since the scan range of the radiographic image in which the resolution has been reduced can be narrowed down in a stepwise manner, the processing time required for estimating the positions of the markers can be shortened. Although the X-ray tube coordinate changes with the accuracy of less than one pixel, but the final estimation of the coordinates of the marker is performed using the radiographic image of the original resolution. Therefore, it is possible to estimate the coordinates of the markers with a high degree of accuracy, and as a result, it is possible to estimate the X-ray tube coordinate with a high degree of accuracy.

Although the present embodiment has been described based on embodiments and modifications, the above-described embodiments are for facilitating the comprehension of the present embodiment, and are not intended to limit the embodiment. This aspect may be modified and improved without departing from the spirit and scope thereof, and the present aspect includes equivalents thereof. In addition, unless the technical feature is described as essential in this specification, the technical feature can be appropriately deleted.

### DESCRIPTION OF SYMBOLS

1: Radiographic image capturing apparatus
2: X-ray tube
3: Position change mechanism
4: Detector
5: Phantom
6: Radiographic image processing apparatus
7: Imaging control unit
21: Control unit
23: Acquisition unit
24: Generation unit
25: Search unit
26: Position identification unit
27: Position estimation unit
29: Storage unit
30: Memory
31: Radiographic image
32: Low-resolution image
33: Detection condition
34: Input I/F
35: Output I/F
36: Communication I/F

1. A radiographic image processing apparatus comprising:
an acquisition unit configured to acquire a radiographic image reflecting a plurality of markers:
a generation unit configured to generate a low-resolution image in which a resolution of the radiographic image has been reduced;
a position identification unit configured to identify respective positions of the plurality of markers in the low-resolution image, based on a characteristic of the plurality of markers; and
a position estimation unit configured to estimate positions of the plurality of markers in the radiographic image by searching for positions on the radiographic image corresponding to the respective positions of the plurality of markers in the low-resolution image.

2. The radiographic image processing apparatus as recited in claim 1, further comprising:
a search unit configured to search for a region of interest reflecting the plurality of markers in the low-resolution image, based on the characteristic of the plurality of markers.

3. The radiographic image processing apparatus as recited in claim 2,
wherein the search unit narrows down a scan region with respect to the low-resolution image in a stepwise manner, based on the characteristic of the plurality of markers.

4. The radiographic image processing apparatus as recited in claim 2,
wherein the search unit identifies a temporary region of interest including a region reflecting the plurality of markers in the low-resolution image and identifies the region of interest reflecting the plurality of markers from the temporary region of interest, based on the characteristic of the plurality of markers.

5. The radiographic image processing apparatus as recited in claim 2,
wherein the position identification unit identifies respective barycentric coordinates of the plurality of markers included in the region of interest as the respective positions of the plurality of markers in the low-resolution image, based on the characteristic of the plurality of markers.

6. A radiographic image processing method to be performed by a radiographic image processing apparatus, the method comprising:
acquiring a radiographic image reflecting a plurality of markers;
generating a low-resolution image in which a resolution of the radiographic image has been reduced;
identifying respective positions of the plurality of markers in the low-resolution image, based on the characteristic of the plurality of markers; and
estimating positions of the plurality of markers in the radiographic image by searching for positions on the radiographic image corresponding to the respective positions of the plurality of markers in the low-resolution image.

7. A radiographic image processing program for making a computer execute processing, the processing comprising:
acquiring a radiographic image reflecting a plurality of markers;
generating a low-resolution image in which a resolution of the radiographic image has been reduced;
identifying respective positions of the plurality of markers in the low-resolution image, based on the characteristic of the plurality of markers; and
estimating positions of the plurality of markers in the radiographic image by searching for positions on the radiographic image corresponding to the respective positions of the plurality of markers in the low-resolution image.
